(19) United States
(12) Patent Application Publication
(10) Pub. No.: US 2016/0074287 A1
(43) Pub. Date: Mar. 17, 2016

(54) INFANT MEDICINE FEEDER

(71) Applicant: Tianjin Kelin Mold Technic Co., Ltd., Tianjin (CN)

(72) Inventor: Kecheng WANG, Tianjin (CN)

(21) Appl. No.: 14/676,842

(22) Filed: Apr. 2, 2015

Related U.S. Application Data
(63) Continuation-in-part of application No. PCT/CN2014/091029, filed on Nov. 13, 2014.

(30) Foreign Application Priority Data
Sep. 16, 2014 (CN) 201420531224.0

(51) Int. Cl. A61J 17/00; A61M 5/315

(52) U.S. Cl. CPC A61J 17/006; A61M 5/315

(57) ABSTRACT

An infant medicine feeder, including a pacifier, a protection plate, a connection disk, and a syringe. The protection plate is connected to the tail of the pacifier. The protection plate is connected to the syringe. The protection plate includes two rectangle holes, and one end of each of the rectangle holes is provided with a step. The pacifier, the protection plate, the connection disk, and the syringe are connected in series through socketing of corresponding axle holes; one surface of the connection disk facing the protection plate is provided with two elastic snaps corresponding to the two rectangle holes. The two elastic snaps of the connection disk are snapped in the steps. A liquid channel is formed from the cavity of the syringe to the forepart of the pacifier, and the outlet of the liquid channel is disposed at the flat surface of the forepart of the pacifier.

### CROSS-REFERENCE TO RELATED APPLICATIONS

This application is a continuation-in-part of International Patent Application No. PCT/CN2014/091029 with an international filing date of Nov. 13, 2014, designating the United States, now pending, and further claims priority benefits to Chinese Patent Application No. 201420531224.0 filed Sep. 16, 2014. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND OF THE INVENTION

1. Field of the Invention

The invention relates to an infant medicine feeder.

2. Description of the Related Art

A typical infant medicine administration mode includes dissolving medicine in a water bottle or a milk bottle and feeding the solution to the infant. However, the administration mode is time-consuming, and the infant will reject the medicine after smelling the bitterness thereof. Although there is a special infant medicine feeder on the market, it has a complex structure thus being difficult to disassemble and clean.

### SUMMARY OF THE INVENTION

In view of the above-described problems, it is one objective of the invention to provide an infant medicine feeder that has a simple structure and is easy to disassemble and clean.

To achieve the above objective, in accordance with one embodiment of the invention, there is provided an infant medicine feeder, comprising a pacifier; a protection plate; a connection disk; and a syringe. The protection plate is connected to a tail of the pacifier and is configured to prevent the infant medicine feeder from being swallowed. The protection plate is connected to the syringe via the connection disk. The protection plate comprises two rectangle holes, and one end of each of the rectangle holes is provided with a step. The pacifier, the protection plate, the connection disk, and the syringe are connected in series through socketing of corresponding axle holes; one surface of the connection disk facing the protection plate is provided with two elastic snaps corresponding to the two rectangle holes. The two elastic snaps of the connection disk are snapped in the steps of the two rectangle holes of the protection plate; a liquid channel is formed from a cavity of the syringe to a forepart of the pacifier, and an outlet of the liquid channel is disposed at a flat surface of the forepart of the pacifier.

In a class of this embodiment, the syringe comprises a piston; the piston is connected to a push rod, and a wall of the syringe comprises calibration lines.

In a class of this embodiment, the protection plate comprises lightening holes.

In a class of this embodiment, two end surfaces of the connection disk are provided with pipe joints for socketing of the protection plate and the syringe.

In a class of this embodiment, a first end surface of the protection plate facing the pacifier comprises a groove for receiving the tail of the pacifier; and a second end surface of the protection plate facing the connection disk comprises a circular recess for receiving an upper pipe joint of the connection disk.

Compared to the prior art, advantages of the invention are summarized as follows. The infant medicine feeder of the invention comprises a pacifier which is basically the same as a common pacifier in structure. The pacifier can bring comfortable feelings for infants. When it is time for administration, the infant medicine feeder is substituted for the common pacifier without the awareness of the infant, and then push the push rod so that the medicine is fed to the infant, which is convenient and timesaving.

### BRIEF DESCRIPTION OF THE DRAWINGS

FIG. 1 is an exploded view of an infant medicine feeder according to one embodiment of the invention;

FIG. 2 shows a connection of rectangle holes of a protection plate and elastic snaps of a connection disk according to one embodiment of the invention; and

FIG. 3 is a schematic diagram of an infant medicine feeder according to one embodiment of the invention.

In the drawings, the following reference numbers are used: 1. Pacifier; 11. Outlet (medicine feeding hole) of liquid channel; 2. Protection plate; 21. Rectangle hole; 22. Step; 23. Lightening holes; 24. Groove; 3. Connection disk; 31. Elastic snap; 4. Syringe; 41. Piston; 42. Push rod.

### DETAILED DESCRIPTION OF THE EMBODIMENTS

For further illustrating the invention, experiments detailing a permanent magnet rotor are described below. It should be noted that the following examples are intended to describe and not to limit the invention.

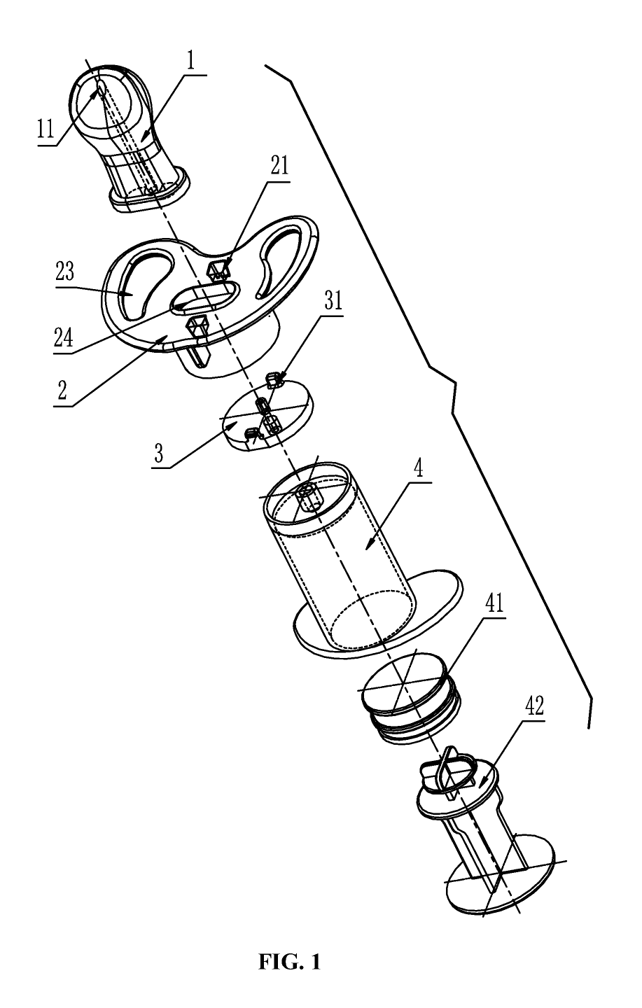

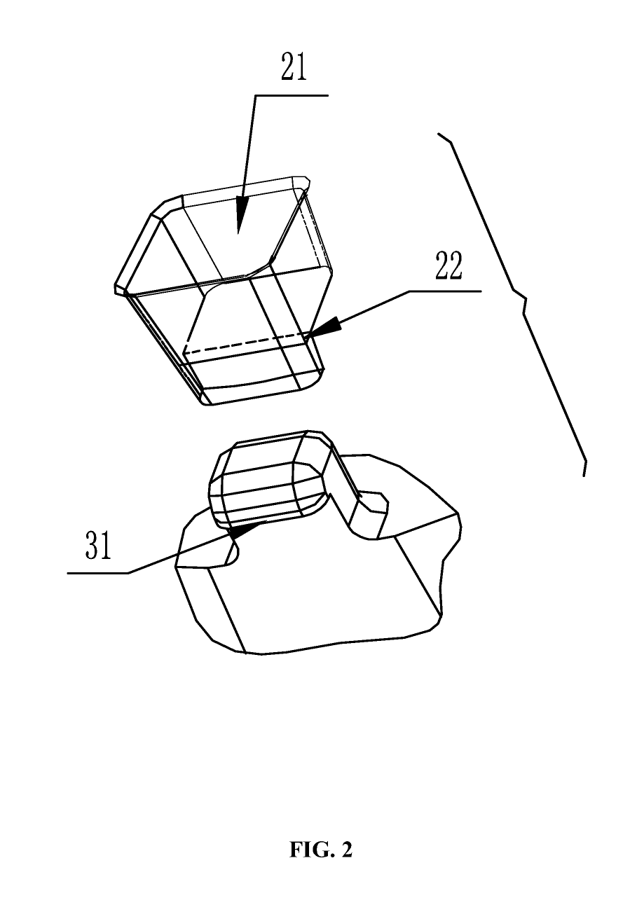

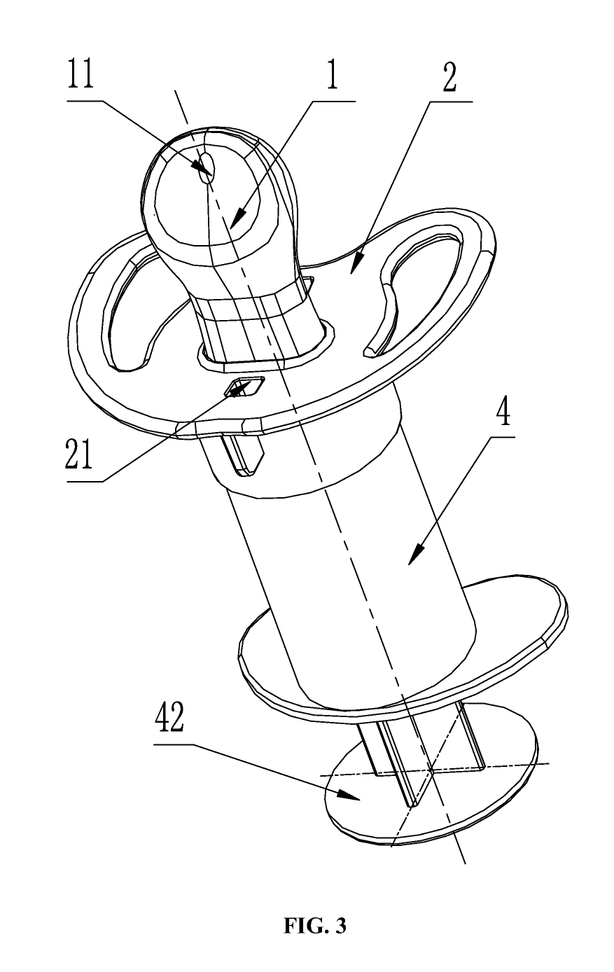

As shown in FIGS. 1 and 3, the invention provides an infant medicine feeder comprising a pacifier 1 and a protection plate 2 which is connected to a tail of the pacifier. The protection plate 2 is configured to prevent the infant medicine feeder from being swallowed by the infant and comprises lightening holes 23. The protection plate 2 is connected to a syringe 4 via a connection disk 3. The protection plate 2 comprises two rectangle holes 21, and one end of each of the rectangle holes 21 is provided with a step 22. The syringe 4 comprises a piston 41, the piston 41 is connected to a push rod 42, and a wall of the syringe 4 comprises calibration lines. Two end surfaces of the connection disk 3 are provided with pipe joints for the socketing of the protection plate 2 and the syringe 4. A first end surface of the protection plate 2 facing the pacifier 1 comprises a groove 24 for receiving the tail of the pacifier 1; and a second end surface of the protection plate 2 facing the connection disk 3 comprises a circular recess not shown in the drawings for receiving an upper pipe joint of the connection disk 3. The pacifier 1, the protection plate 2, the connection disk 3, and the syringe 4 are connected in sequence through socketing of axle holes; one surface of the connection disk 3 facing the protection plate 2 is provided with two elastic snaps 31 corresponding to the two rectangle holes 21. As shown in FIG. 2, the two elastic snaps 31 is clasped on the steps of the two rectangle holes 21. Thus, the connection disk 3 connects the pacifier 1, the protection plate 2 and the syringe 4 as a whole. A liquid channel is formed from a cavity of the syringe 4 to a forepart of the pacifier 1, and an outlet 11 medicine feeding hole of the liquid channel is disposed at a flat surface of the forepart of the pacifier 1.

The infant medicine feeder is assembled as follows. Firstly, the pacifier 1 is inserted into the protection plate 2, and the pipe joint at one side of the connection disk 3 is inserted into the protection plate 2. The two elastic snaps 31 of the connection disk 3 are snapped in the steps of the two rectangle holes 21 of the protection plate 2, whereby clasping the pacifier 1. The piston 41 and the push rod 42 are assembled into the syringe 4 to form an injection structure. A certain amount of medicine is sucked. Thereafter, the bottom hole of the syringe 4 is inserted into a pipe joint at the other side of the connection disk 3 so as to connect the syringe 4 to the protection plate 2. Prior to administration, an infant is sucking a common pacifier for acquiring comfortable feelings. When it is time for administration, the infant medicine feeder is substituted for the common pacifier without the awareness of the infant, and then push the push rod so that the medicine is fed to the infant, which is convenient and timesaving.

The outlet of the liquid channel is disposed at the flat surface of the forepart of the pacifier 1 which tends to contact the tongue of the infant, so that, during administration, the medicine is first sprayed on the tongue of the infant, which prevents the medicine from being directly injected into the throat or trachea thereby avoiding the choke.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. An infant medicine feeder, comprising:
a) a pacifier;
b) a protection plate;
c) a connection disk; and
d) a syringe;
wherein
the protection plate is connected to a tail of the pacifier and is configured to prevent the infant medicine feeder from being swallowed;
the protection plate is connected to the syringe via the connection disk;
the protection plate comprises two rectangle holes, and one end of each of the rectangle holes is provided with a step;
the pacifier, the protection plate, the connection disk, and the syringe are connected in series through socketing of corresponding axle holes;
one surface of the connection disk facing the protection plate is provided with two elastic snaps corresponding to the two rectangle holes; the two elastic snaps of the connection disk are snapped in the steps of the two rectangle holes of the protection plate;
a liquid channel is formed from a cavity of the syringe to a forepart of the pacifier, and an outlet of the liquid channel is disposed at a flat surface of the forepart of the pacifier.

2. The infant medicine feeder of claim 1, wherein the syringe comprises a piston; the piston is connected to a push rod, and a wall of the syringe comprises calibration lines.

3. The infant medicine feeder of claim 1, wherein the protection plate comprises lightening holes.

4. The infant medicine feeder of claim 3, wherein two end surfaces of the connection disk are provided with pipe joints for socketing of the protection plate and the syringe.

5. The infant medicine feeder of claim 3, wherein a first end surface of the protection plate facing the pacifier comprises a groove for receiving the tail of the pacifier;
and a second end surface of the protection plate facing the connection disk comprises a circular recess for receiving an upper pipe joint of the connection disk.
